(19) United States
(12) Patent Application Publication
(10) Pub. No.: US 2024/0423593 A1
(43) Pub. Date: Dec. 26, 2024

(54) ULTRASOUND DIAGNOSTIC SYSTEM, STORAGE MEDIUM, ULTRASOUND IMAGE DISPLAY TERMINAL, AND ULTRASOUND DIAGNOSTIC APPARATUS

(71) Applicant: Konica Minolta, Inc., Tokyo (JP)

(72) Inventor: Takashi SAKAI, Yokohama-shi (JP)

(21) Appl. No.: 18/661,837

(22) Filed: May 13, 2024

(30) Foreign Application Priority Data
Jun. 21, 2023 (JP) 2023-101399

(51) Int. Cl. A61B 8/00; A61B 8/08

(52) U.S. Cl. CPC A61B 8/565; A61B 8/461; A61B 8/5207; A61B 8/54

(57) ABSTRACT

An ultrasound diagnostic system includes: an ultrasound diagnostic apparatus that generates image data; and an ultrasound image display terminal that is a separate unit from the ultrasound diagnostic apparatus and displays an ultrasound image based on the image data. The ultrasound diagnostic apparatus and the ultrasound image display terminal are connected with one another via a wireless network. The ultrasound diagnostic system further includes: a transmitter that transmits the image data from the ultrasound diagnostic apparatus to the ultrasound image display terminal; and a hardware processor that outputs delay information indicating that a transmission delay of the image data is occurring.

### BACKGROUND OF THE INVENTION

### Technical Field

The present invention relates to an ultrasound diagnostic system, a storage medium, an ultrasound image display terminal, and an ultrasound diagnostic apparatus.

### Description of Related Art

An ultrasound diagnostic apparatus can obtain the state of organ and tissue as an ultrasound image by a simple operation of applying an ultrasound probe to the body surface of a patient or through the body cavity thereof. This ultrasound diagnostic apparatus is also used in a puncture process for detecting the position of the tip of a puncture needle that has been inserted into a subject.

It is desirable that the puncture process is performed in a state where the direction in which an operator views a display for the ultrasound image and the entering direction of the puncture needle are made to coincide with each other. Thus, the operator does not need to turn his/her head to the side when viewing the display, and therefore it is possible to prevent the probe from being shaken and the entering direction of the puncture needle from being shifted.

As the display, an easily portable tablet terminal or the like is used. In this case, the ultrasound diagnostic apparatus and the display are wirelessly connected by Wi-Fi® or the like. On the screen of the wirelessly connected display, the ultrasound image same as that of the ultrasound diagnostic apparatus is displayed. With the wireless configuration, convenience such as operability and layout can be improved.

In Japanese Translation of PCT International Publication 2008-513084, there are disclosed an ultrasound imaging system and an ultrasound imaging apparatus that includes wireless displays that are separate from the ultrasound imaging system. The IEEE standard 802.15.3 is used for transmission of images from the ultrasound imaging system to each of the wireless displays.

### SUMMARY OF THE INVENTION

However, in the conventional technology, when an image is transmitted from the ultrasound imaging system to each display, a transmission delay may occur due to the wireless configuration. Along with this, a delay may occur in an ultrasound image displayed on the screen of the display. For example, if a delay occurs in image display when the nerve block is punctured, a medical error such as a nerve injury may occur.

Therefore, in order to solve the above-described problems, objects of the present invention include providing an ultrasound diagnostic system, a storage medium storing a program, an ultrasound image display terminal, and an ultrasound diagnostic apparatus that are capable of preventing a medical error due to occurrence of a transmission delay even if the ultrasound diagnostic apparatus and the ultrasound image display terminal are connected wirelessly.

To achieve at least one of the abovementioned objects, according to an aspect of the present invention, an ultrasound diagnostic system reflecting one aspect of the present invention includes:
an ultrasound diagnostic apparatus that generates image data; and
an ultrasound image display terminal that is a separate unit from the ultrasound diagnostic apparatus and displays an ultrasound image based on the image data;
wherein the ultrasound diagnostic apparatus and the ultrasound image display terminal are connected with one another via a wireless network, and
wherein the ultrasound diagnostic system further comprises:
a transmitter that transmits the image data from the ultrasound diagnostic apparatus to the ultrasound image display terminal; and
a hardware processor that outputs delay information indicating that a transmission delay of the image data is occurring.

According to an aspect of the present invention, a non-transitory computer-readable storage medium reflecting one aspect of the present invention stores a program that causes an ultrasound image display terminal that is a separate unit from an ultrasound diagnostic apparatus and is connected with the ultrasound diagnostic apparatus via a wireless network, the ultrasound diagnostic apparatus generating image data, to perform:
receiving the image data transmitted from the ultrasound diagnostic apparatus;
displaying an ultrasound image based on the image data; and
outputting delay information indicating that a transmission delay of the image data is occurring.

According to an aspect of the present invention, a non-transitory computer-readable storage medium reflecting one aspect of the present invention stores a program that causes an ultrasound diagnostic apparatus that is a separate unit from an ultrasound image display terminal, is connected with the ultrasound image display terminal via a wireless network, and generates image data, the ultrasound image display terminal displaying an ultrasound image, to perform:
transmitting the image data to the ultrasound image display terminal; and
outputting, to the ultrasound image display terminal, time information for calculating delay information indicating that a transmission delay of the image data is occurring.

According to an aspect of the present invention, an ultrasound image display terminal reflecting one aspect of the present invention is a separate unit from an ultrasound diagnostic apparatus and is connected with the ultrasound diagnostic apparatus via a wireless network, the ultrasound diagnostic apparatus generating image data, and includes:
a receiver that receives the image data transmitted from the ultrasound diagnostic apparatus;
a display that displays an ultrasound image based on the image data; and
a hardware processor that outputs delay information indicating that a transmission delay of the image data is occurring.

According to an aspect of the present invention, an ultrasound diagnostic apparatus reflecting one aspect of the present invention is a separate unit from an ultrasound image display terminal, is connected with the ultrasound image display terminal via a wireless network, and generates image data, the ultrasound image display terminal displaying an ultrasound image, and includes:
a transmitter that transmits the image data to the ultrasound image display terminal; and
a hardware processor that outputs, to the ultrasound image display terminal, time information for calculating delay information indicating that a transmission delay of the image data is occurring.

### BRIEF DESCRIPTION OF THE DRAWINGS

The advantages and features provided by one or more embodiments of the present invention will become more fully understood from the detailed description given hereinafter and the appended drawings which are given by way of illustration only, and thus are not intended as a definition of the limits of the present invention, and wherein:

FIG. 1 shows an example of a schematic configuration of an ultrasound diagnostic system according to an embodiment;

FIG. 2 is an illustration for explaining delay of an ultrasound image displayed on an ultrasound image display terminal according to the embodiment;

FIG. 3 is a block diagram of an ultrasound diagnostic apparatus according to the embodiment;

FIG. 4 is a block diagram of the ultrasound image display terminal according to the embodiment;

FIG. 5 is a flowchart showing an example of operation of the ultrasound diagnostic apparatus at the time of pre-scanning and a puncture process according to the embodiment;

FIG. 6 is a flowchart showing an example of operation of the ultrasound image display terminal at the time of the pre-scanning and the puncturing according to the embodiment;

FIG. 7A shows an example of a screen of a display part of the ultrasound image display terminal in a case where transmission time according to the embodiment is within an allowable range;

FIG. 7B shows an example of the screen of the display part of the ultrasound image display terminal in a case where the transmission time according to the embodiment exceeds the allowable range; and

FIG. 8 is a sequence diagram showing a flow at the time of the pre-scanning and the puncture process in the ultrasound diagnostic apparatus and the ultrasound image display terminal according to the embodiment.

### DETAILED DESCRIPTION

Hereinafter, one or more preferred embodiments of the present disclosure will be described with reference to the accompanying drawings. However, the scope of the disclosure not limited to the disclosed embodiments.

### Configuration Example of Ultrasound Diagnostic System 1

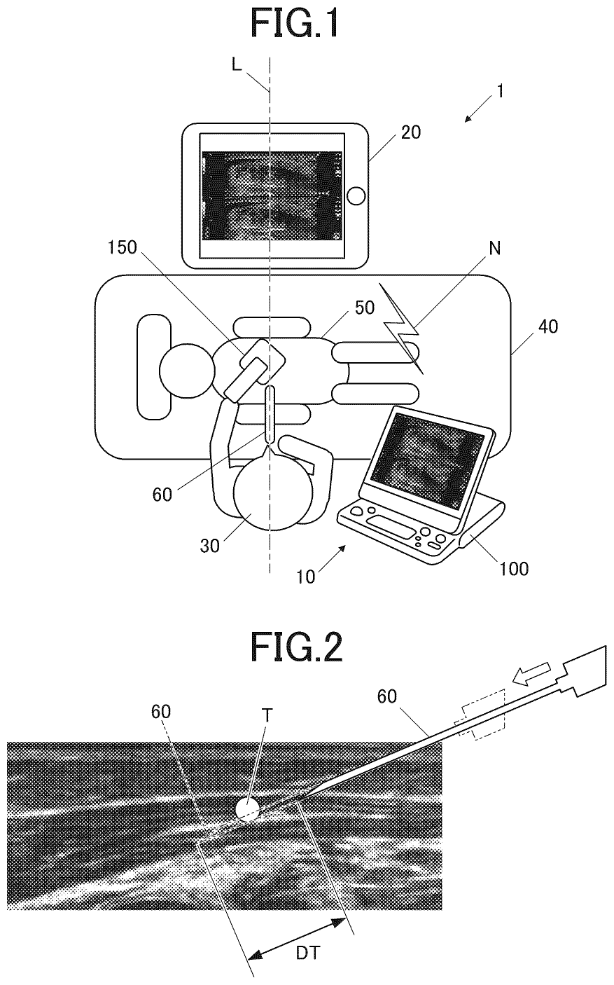

FIG. 1 shows an example of a schematic configuration of an ultrasound diagnostic system 1.

The ultrasound diagnostic system 1 includes an ultrasound diagnostic apparatus 10 and an ultrasound image display terminal 20 that is a separate unit from the ultrasound diagnostic apparatus 10. Hereinafter, the ultrasound diagnostic apparatus 10 may be referred to as the diagnostic apparatus 10, and the ultrasound image display terminal 20 may be referred to as the display terminal 20. The diagnostic apparatus 10 and the display terminal 20 are communicably connected to each other by a wireless network N. Examples of the wireless network N include a wireless LAN such as Wi-Fi, short-range wireless communication such as Bluetooth®, mobile communication, and the like.

The diagnostic apparatus 10 includes an ultrasound diagnostic apparatus main body 100 and an ultrasound probe 150 connected via a cable (not shown). Hereinafter, the ultrasound diagnostic apparatus main body 100 may be referred to as the apparatus main body 100. A user 30 such as a doctor presses the ultrasound probe 150 against the body surface of a patient 50 lying on his/her back on a bed 40. The ultrasound probe 150 transmits ultrasound waves into the body of the patient 50 and receives echo signals reflected at the boundaries between tissues having different acoustic impedances in the body.

The apparatus main body 100 generates ultrasound image data of the internal state of the body where a puncture process is performed based on the echo signal received by the ultrasound probe 150. The apparatus main body 100 wirelessly transmits the generated ultrasound image data to the display terminal 20, and displays a tomographic ultrasound image on a display part 114. The display terminal 20 displays a tomographic ultrasound image based on the ultrasound image data transmitted from the ultrasound diagnostic apparatus 10 on a screen. In this way, the puncture process is performed while the ultrasound image being displayed on the diagnostic apparatus 10 is mirrored on the display terminal 20.

In the present embodiment, the puncture process is performed on the patient 50 in a state where the viewing direction in which the user 30 views the ultrasound image on the display terminal 20 and the entering direction of a puncture needle 60 are aligned with each other. In other words, the user 30, the puncture needle 60 held by the user, and the display terminal 20 are positioned on the same straight line L. In the present embodiment, such a puncture process is referred to as inline puncture.

Here, when the ultrasound image data is wirelessly transmitted from the diagnostic apparatus 10 to the display terminal 20, a transmission delay that exceeds a certain range may occur. The causes of the transmission delay are considered to be a process delay, a communication delay, and the like of an arithmetic unit such as a CPU on the diagnostic apparatus 10 side that encodes the ultrasound image data or the like. As other causes of the transmission delay, a process delay, a communication delay, and the like of an arithmetic unit such as a CPU on the display terminal 20 side that decodes the ultrasound image data or the like are considered. When the transmission delay of the ultrasound image data occurs, a delay occurs in the ultrasound image displayed on the display terminal 20. As a result, a shift occurs between the puncture by the user 30 and the movement of the ultrasound image displayed on the display terminal 20 visually recognized by the user 30.

FIG. 2 is a diagram for explaining the delay of the ultrasound image displayed on the display terminal 20. As illustrated in FIG. 2, the actual puncture needle 60 has advanced to a position indicated by a broken line that has passed through a target T in the body of the patient. However, the puncture needle 60 is displayed at a position indicated by a solid line in front of the target T on the screen due to the transmission delay. Since the user moves the puncture needle 60 only during the puncture process, it is difficult for the user to recognize the delay of the ultrasound image. In a case where a delay time DT is large and exceeds an allowable range, the display delay of the image is also large, and thus a medical error is likely to occur. In the present embodiment, when the transmission delay occurs, the user is alerted. Specifically, the display terminal 20 displays, on the screen, delay information indicating that the transmission delay is occurring.

### Configuration Example of Ultrasound Diagnostic Apparatus 10

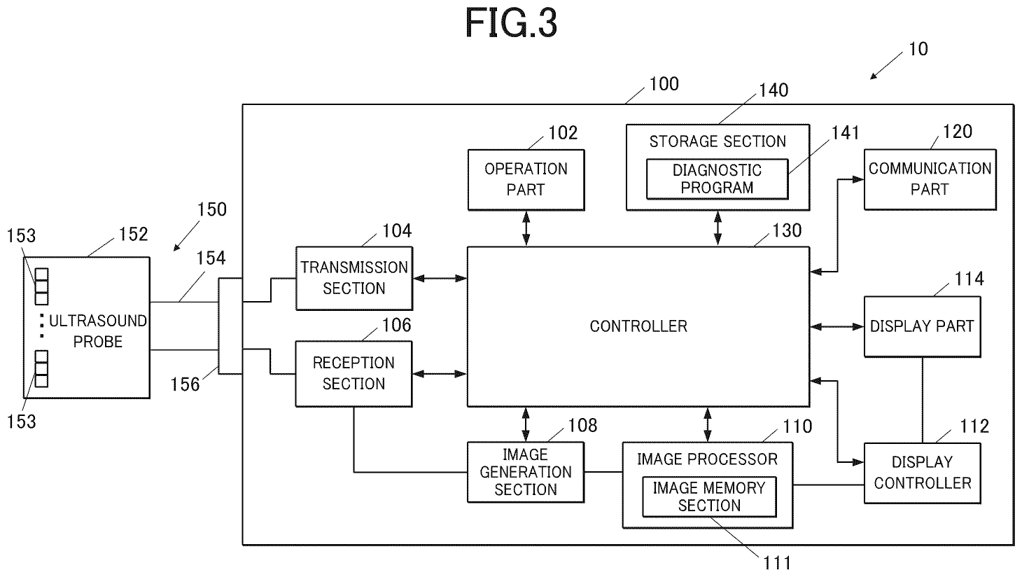

FIG. 3 is a block diagram of the diagnostic apparatus 10 according to the present embodiment.

The diagnostic apparatus 10 is used by a user such as a doctor or a technician in a medical facility such as a hospital, for example. The diagnostic apparatus 10 includes a computer and includes the apparatus main body 100 and the ultrasound probe 150 connected to the apparatus main body 100.

The apparatus main body 100 includes an operation part 102, a transmission section 104, a reception section 106, an image generation section 108, and an image processor 110. The apparatus main body 100 further includes a display controller 112, a display part 114, a communication part 120 (transmitter), a controller 130 (hardware processor), and a storage section 140 (storage).

The operation part 102 receives various input operations from the user, converts the received input operations into electrical signals, and outputs the electrical signals to the controller 130. The operation part 102 includes, for example, a mouse, a keyboard, a trackball, a switch, and a button. The operation part 102 may be, for example, a touch screen integrally combined with a display. The operation part 102 may be, for example, a user interface such as a microphone that receives a voice input. In the present embodiment, the operation part 102 receives, for example, commands for starting and ending pre-scanning and the puncture process. The operation part 102 receives, for example, input of various image parameters such as frequency and depth. The image parameters can be set, for example, at the time of pre-scanning.

Under the control of the controller 130, the transmission section 104 supplies a drive signal, which is an electrical signal, to the ultrasound probe 150. The transmission section 104 includes, for example, a clock generation circuit, a delay circuit, and a pulse generation circuit. The clock generation circuit generates a clock signal that determines a transmission timing and/or a transmission frequency of the drive signal. The delay circuit sets a delay time for each path provided in each oscillator 153 described later, and delays transmission of the drive signal by the set delay time. The delay circuit converges transmission beams formed by the transmission ultrasound waves. The pulse generation circuit generates a pulse signal as a drive signal at a predetermined cycle. For example, the transmission section 104 drives part of the plurality of oscillators 153 being continuous to generate a transmission ultrasound wave. The transmission section 104 scans while shifting the oscillators 153 to drive in the azimuth direction 5 each time it generates the transmission ultrasound wave.

Under the control of the controller 130, the reception section 106 receives a reception signal, which is an electrical signal, from the ultrasound probe 150. The reception section 106 includes, for example, an amplifier, an A/D conversion circuit, and a phasing addition circuit. The amplifier amplifies the reception signal at a preset amplification factor for each path provided in each oscillator 153. The A/D conversion circuit performs analog-digital conversion on the amplified reception signal. The phasing addition circuit gives a delay time to the A/D converted reception signal for each path provided in each oscillator 153 to adjust the time phase, and adds these. The phasing addition circuit generates sound ray data or sound ray signals by phasing addition. Note that the reception section 106 may include an amplifier for amplifying the reception signal.

The image generation section 108 performs envelope detection, logarithmic compression, and the like on the sound ray data supplied from the reception section 106, under the control of the controller 130. The image generation section 108 further performs dynamic range adjustment and/or gain adjustment on the sound ray data to convert the luminance, thereby generating B-mode image data. The B-mode image data is data in which the intensity of a reception signal is represented by luminance, and is tomographic image information regarding a tissue in the subject. The image generation section 108 is not limited to generating the B-mode image data of the B mode. Examples of the other scan modes (image modes) include an A mode, an M mode, and a scan mode using a Doppler method. Examples of the Doppler method include a color Doppler mode and a PWD. The B mode is an abbreviation for Brightness mode. The A mode is an abbreviation for Amplitude mode. The M mode is an abbreviation for Motion mode. The PWD is an abbreviation for Pulsed Wave Doppler.

The image processor 110 performs image processing on the B-mode image data output from the image generation section 108, in accordance with various image parameters being set, under the control of the controller 130. The image processor 110 includes an image memory section 111 constituted by a semiconductor memory such as a DRAM. The DRAM is an abbreviation for Dynamic Random Access Memory. The image processor 110 stores the B-mode image data subjected to the image processing in the image memory section 111 in units of frames under the control of the controller 130. In the present embodiment, the B-mode image data in units of frames may be referred to as ultrasound image data or frame image data. Under the control of the controller 130, the image processor 110 sequentially outputs the ultrasound image data generated as described above to the display controller 112.

The display controller 112 generates an image signal for display by performing coordinate conversion or the like on the received ultrasound image data. under the control of the controller 130. The display controller 112 outputs the generated image signal for display to the display part 114.

The display part 114 is, for example, a display device such as an LCD or an organic EL display. The LCD is an abbreviation for Liquid Crystal Display. The EL is an abbreviation for Electronic Luminescence. Under the control of the controller 130, the display part 114 displays, on the screen, an ultrasound image showing the internal state of the body of the patient based on the image signal for display. The ultrasound image may be a still image or a moving image.

The communication part 120 includes, for example, a communication module or the like including an NIC, a receiver, and a transmitter. The NIC is a Network Interface Card. The communication part 120 transmits, for example, various types of data on ultrasound image data or the like to the display terminal 20 connected thereto via the wireless network N. Note that the communication part 120 functions as a transmitter.

The controller 130 includes a processor such as a CPU, a memory such as a RAM, and the like. The CPU is an abbreviation for Central Processing Unit. The RAM is an abbreviation for Random Access Memory. The CPU reads various process programs stored in the storage section 140, loads the programs to the RAM, and executes various processes in cooperation with the programs. The CPU may be constituted by a single processor or a plurality of processors.

The processor of the controller 130 realizes a transmission step and an output step by executing a diagnostic program 141 stored in the storage section 140 or the like. The transmission step is a function of transmitting the ultrasound image data to the display terminal 20 from the communication part 120. The output step is a function of outputting, to the display terminal 20, time information for calculating delay information indicating that a transmission delay of the ultrasound image data is occurring.

The storage section 140 includes any storage module, such as an HDD, an SSD, a ROM, and/or a RAM. The HDD is an abbreviation for Hard Disk Drive. The SSD is an abbreviation for Solid State Drive. The ROM is an abbreviation for Read Only Memory. The storage section 140 stores, for example, a system program, an application program(s), and various types of data received by the communication part 120. In the present embodiment, the storage section 140 stores the diagnostic program 141 for implementing various functions in ultrasound diagnosis.

As shown in FIG. 3, the ultrasound probe 150 includes a head part 152, a cable 154, and a connector 156. The head part 152 is a member that is located on the tip end side of the ultrasound probe 150 and is pressed against the body surface of the patient 50. The head part 152 is provided with a plurality of oscillators 153 formed of piezoelectric elements. The oscillator(s) 153 transmits an ultrasound wave to the patient 50 based on a drive signal transmitted from the apparatus main body 100, and receives a reflected wave reflected inside the body of the patient 50. The plurality of oscillators 153 may be arranged in, for example, a one-dimensional array or a two-dimensional array (matrix) in the scanning direction. The number of oscillators 153 can be set as appropriate. In the present embodiment, an electronic scanning probe of a linear scanning method is used as the ultrasound probe 150, and ultrasound waves are scanned by the linear scanning method. Other than the linear scanning method, a scanning method such as a convex scanning method or a sector scanning method may be employed.

The cable 154 has one end electrically connected to the head part 152 and the other end electrically connected to the connector 156. The connector 156 is attached to the other end of the cable 154 and is connectable to the apparatus main body 100. The connector 156 is, for example, a plug-type connector, and is detachably attached to a receptacle-type connector of the apparatus main body 100. Note that the communication between the apparatus main body 100 and the ultrasound probe 150 is not limited to wired communication using the cable 154. The communication between the apparatus main body 100 and the ultrasound probe 150 may be, for example, wireless communication such as UWB. The UWB is an abbreviation for Ultra Wide Band.

Note that although the image generation section 108 generates ultrasound image data as the type of image data in the above-described example, this is not a limitation. For example, the image generation section 108 may generate sound ray data, tomographic image data, or the like before being subjected to a predetermined process, and transmit the sound ray data or the like to the display terminal 20. The display terminal 20 generates ultrasound image data by performing a predetermined process on the received sound ray data, tomographic image data, or the like. In this case, a program for processing the sound ray data or the like may be transmitted from the diagnostic apparatus 10 to the display terminal 20. The program for processing the sound ray data or the like may be installed in the display terminal 20 in advance.

### Configuration Example of Ultrasound Image Display Terminal 20

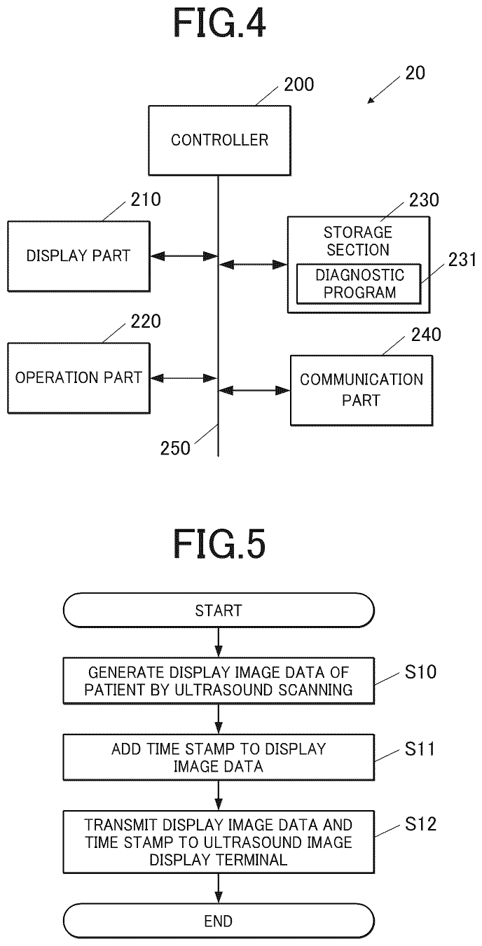

FIG. 4 is a block diagram of the display terminal 20.

The display terminal 20 is a portable terminal and is, for example, a computer such as a tablet, a notebook personal computer, or a display. The display terminal 20 includes a controller 200 (hardware processor), a display part 210 (display), an operation part 220, a storage section 230, and a communication part 240 (receiver). The controller 200, the display part 210, the operation part 220, the storage section 230, and the communication part 240 are connected to each other via, for example, a bus 250.

The controller 200 includes at least a processor such as a CPU and a memory such as a RAM. The CPU loads various programs stored in the storage section 230 into the RAM and executes the programs to implement various functions related to ultrasound diagnosis. The CPU may be constituted by a single processor or a plurality of processors.

The processor of the controller 200 realizes a reception step, a display step, and an output step by executing a diagnostic program 231 or the like stored in the storage section 230 or the like. The reception step is a function of causing the communication part 240 to receive the ultrasound image data transmitted from the diagnostic apparatus 10. The display step is a function of causing the display part 210 to display an ultrasound image based on the ultrasound image data. The output step has a function of an output section that outputs, to the display part 210 or the like, delay information indicating that a transmission delay of the ultrasound image data is occurring.

Note that the controller 200 may display delay information on the display part 210 when a predetermined condition is satisfied. An example of the predetermined condition is that a display button for determining whether to display the delay information is selected. Another example of the predetermined condition is that the puncture mode is selected as the operation mode.

The diagnostic program 231 for realizing the reception step, the display step, and the output step may be stored, for example, in the storage section 140 of the diagnostic apparatus 10. In this case, the controller 130 of the diagnostic apparatus 10 functions as an output section, and outputs the diagnostic program 231 to the display terminal 20 via the wireless network N. The display terminal 20 receives the diagnostic program 231 or the like transmitted from the diagnostic apparatus 10, and installs the received diagnostic program 231.

The display part 210 displays an ultrasound image based on the ultrasound image data received from the diagnostic apparatus 10, a GUI for receiving various input operations from the user, and the like. The display part 210 is a display such as a liquid crystal display or an organic EL display. In the present embodiment, the display part 210 displays delay information when a transmission delay occurs at the time of transmission of ultrasound image data.

The operation part 220 receives instructions corresponding to various input operations from a user, converts the received instructions into operation signals, and outputs the operation signals to the controller 200. The operation part 220 includes, for example, a mouse, a keyboard, a switch, and a button. The operation part 220 may be, for example, a touch screen integrally combined with a display. The operation part 220 may be, for example, a user interface such as a microphone that receives a voice input.

The storage section 230 stores, for example, a system program, an application program(s), and various types of data. The storage section 230 includes any storage module, such as an HDD, an SSD, a ROM, and/or a RAM. In the present embodiment, the storage section 230 stores the diagnostic program 231, ultrasound image data received from the diagnostic apparatus 10, a time stamp described later, and the like.

The communication part 240 includes, for example, a wireless communication module or the like including an NIC, a receiver, and a transmitter. The communication part 240 receives various data such as ultrasound image data transmitted from the diagnostic apparatus 10 via the wireless network N. Note that the communication part 240 functions as a receiver.

### Operation Example of Ultrasound Diagnostic Apparatus 10

FIG. 5 is a flowchart illustrating an example of operation of the diagnostic apparatus 10 at the time of pre-scanning and the puncture process. In the present embodiment, the flow of processes on the diagnostic apparatus 10 side is common at the time of the pre-scan and the puncture process.

The controller 130 acquires display image data of the patient 50 by ultrasound scanning with the ultrasound probe 150 (Step S10).

The controller 130 generates a time stamp for the acquired display image data, and adds the generated time stamp to the display image data (Step S11). The time stamp is an example of time information and includes a transmission time at which the display image data is transmitted from the diagnostic apparatus 10 to the display terminal 20. The time stamp can be acquired, for example, by a time measurer (not illustrated) which measures time.

The controller 130 transmits the display image data and the time stamp added to the display image data to the display terminals 20 (Step S12).

### Operation Example of Ultrasound Image Display Terminal 20

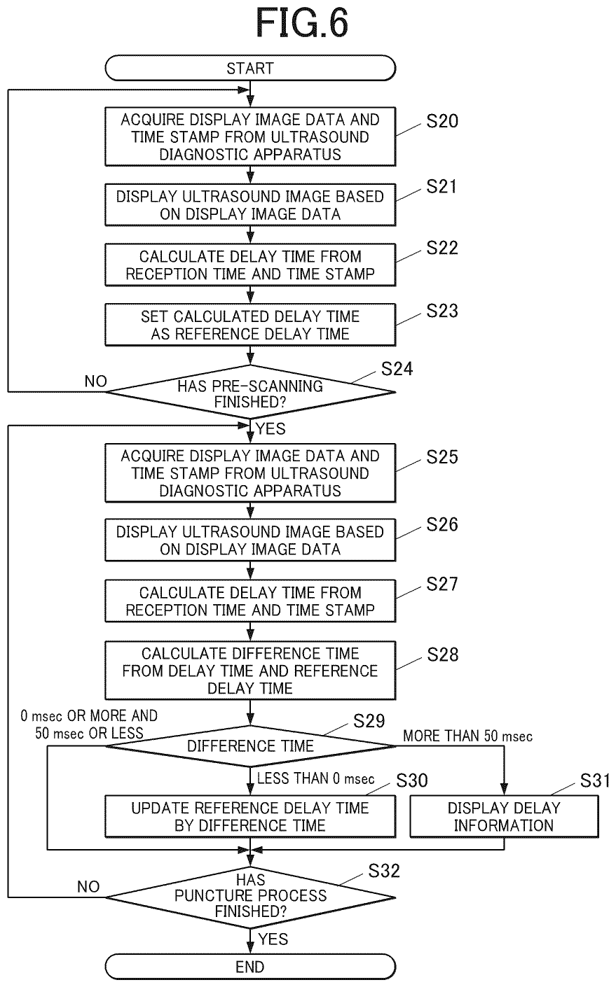

FIG. 6 is a flowchart illustrating an example of operation of the display terminal 20 at the time of pre-scanning and puncturing Note that in the present embodiment, the pre-scanning and the puncture process are performed successively.

First, pre-scanning is performed. The communication part 240 receives display image data and a time stamp from the diagnosis apparatus 10. The controller 200 acquires the display image data and the time stamp received by the communication part 240 (Step S20). At the time, the controller 200 acquires a reception time at which the display image data was received. The reception time can be acquired by, for example, a time measurer (not illustrated) that measures time.

The controller 200 causes the display part 210 to display an ultrasound image based on the received display image data on the screen of the display part 210 (Step S21).

The controller 200 calculates a delay time using the reception time, at which the display image data was received, and the time stamp received from the diagnostic apparatus 10 (Step S22). Specifically, the controller 200 calculates and acquires the delay time by subtracting the stamped time indicated by the time stamp from the reception time.

The controller 200 sets the calculated delay time as a reference delay time and stores the reference delay time in, for example, the storage section 230 (Step S23). If the reference delay time has already been set, the controller 200 rewrites it by the currently calculated delay time as a new reference delay time (i.e., updates the reference delay time). The controller 200 functions as an acquisition section that acquires the reference delay time information at a first time. The stamped time indicated by the time stamp corresponds to the first time.

The reference delay time can be set in accordance with any of the following rules.
(1) The delay time of each frame is calculated, and the reference delay time is the delay time of the latest frame.
(2) If the delay time of the latest frame is smaller than the set reference delay time, the reference delay time is the delay time of the latest frame.
(3) The reference delay time is the average of the delay times of frames acquired within a certain period of time.
(4) The reference delay time is the delay time of a frame acquired at a timing specified by the user or a timing at which the user operates the apparatus.

The controller 200 determines whether the pre-scanning has finished (Step S24). For example, when the user selects the function to display the puncture needle in an emphasized manner, the controller 200 may determine that the pre-scanning has finished and the puncture process has been started. When an operation to end the pre-scanning is made, for example, a pre-scanning end button is selected, the controller 200 may determine that the pre-scanning has finished. The controller 200 may determine that the pre-scanning has finished when an operation to start the puncture process is made, for example, a puncture start button is selected. In the pre-scanning, movement of the ultrasound probe 150 becomes large, and the difference between frames also becomes large. Therefore, when the difference between frames becomes smaller than a predetermined threshold, the controller 200 may determine that the pre-scanning has finished.

When determining that the pre-scanning has not finished. the controller 200 returns to Step S20. In this case, the controller 200 executes the above-described processes on the display image data of the next frame. On the other hand, when determining that the pre-scanning is not being performed, that is, the pre-scanning has finished, the controller 200 proceeds to Step S25.

Subsequently, the puncture process is performed. The communication part 240 receives display image data and a time stamp of the display image data from the diagnosis apparatus 10. The controller 200 acquires the display image data and the time stamp received by the communication part 240 (Step S25). At this time, the controller 200 acquires the reception time at which the display image data was received. The reception time can be acquired by, for example, the time measurer (not illustrated) that keeps time.

The controller 200 causes the display part 210 to display an ultrasound image based on the received display image data on the screen of the display part 210 (Step S26).

The controller 200 calculates a delay time using the reception time, at which the display image data was received, and the time stamp received from the diagnostic apparatus 10 (Step S27). Specifically, the controller 200 calculates and acquires the delay time by subtracting the stamped time indicated by the time stamp from the reception time. The controller 200 functions as an acquisition section that acquires the delay time information at a second time. The stamped time indicated by the time stamp corresponds to the second time.

The controller 200 calculates a relative difference time using the calculated delay time and the reference delay time acquired by the pre-scanning (Step S28). Specifically, the controller 200 calculates the difference time by subtracting the reference delay time from the delay time. Note that the controller 200 functions as a calculation section that calculates, based on the delay time and the reference delay time, a difference time indicating whether the transmission delay is occurring.

The controller 200 determines within which range the calculated difference time falls, the range of less than 0 msec, the range of 0 msec or more and 50 msec or less, or the range of more than 50 msec (Step S29). Note that the controller 200 functions as a determination section that determines whether a transmission delay is occurring.

If the calculated difference time is in the range of 0 msec or more and 50 msec or less, the controller 200) proceeds to Step S32. In this case, the controller 200 determines that a significant transmission delay of the display image data is not occurring, and the difference time is within an allowable range.

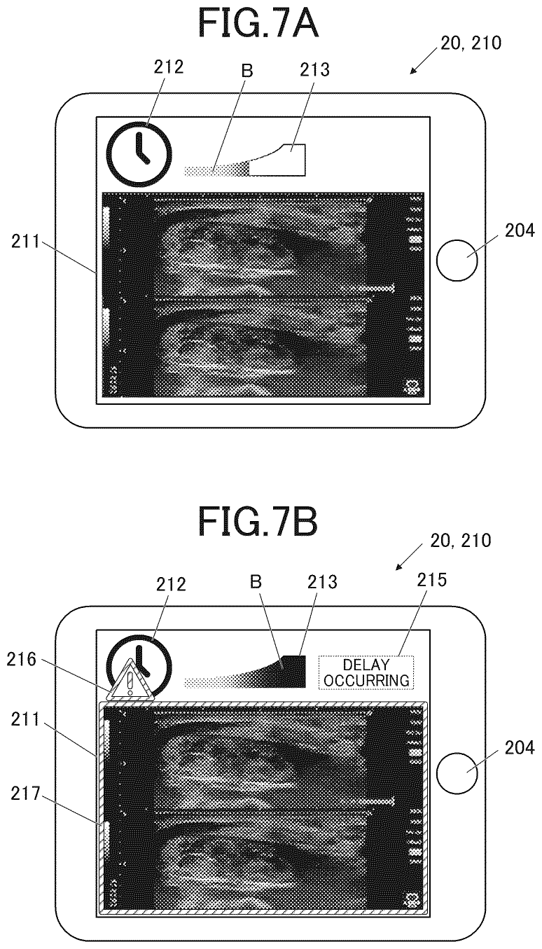

FIG. 7A illustrates an example of the screen of the display part 210 of the display terminal 20 when the transmission delay is within the allowable range. That is, FIG. 7A illustrates the screen of the display part 210 at the normal time (normal state).

A display area 211 in which an ultrasound image is displayed is provided on the screen of the display part 210. Above the display area 211, a clock icon 212 indicating the time and a level meter 213 indicating the magnitude of a transmission delay are provided. A bar B in the level meter 213 becomes longer or shorter according to the degree of the transmission delay. Specifically, when the difference time is within the allowable range, the bar B of the level meter 213 moves, for example, in an area on the left side of the center. In this case, since it is not necessary to call the user's attention, the bar B of the level meter 213 may be displayed in, for example, blue or green.

Note that although the level meter 213 is formed using the bar B in FIG. 7A, it is not limited to this. For example, the bar B of the level meter 213 may be changed to a needle. Further, instead of the level meter 213, the time indicating the transmission delay may be digitally displayed by a number(s) or the like.

If the calculated difference time is less than 0 msec, the controller 200 updates the reference delay time by the difference time (Step S30). Specifically, if the difference time is relatively short as compared with the set reference delay time, the controller 200 sets the difference time that is the minimum as the reference delay time. In this case, since almost no transmission delay is occurring, the screen at the normal time illustrated in the FIG. 7A is displayed on the display part 210.

If the calculated difference time exceeds 50 msec, the controller 200 proceeds to Step S31. In this case, the controller 200 determines that a transmission delay is occurring in the ultrasound image to be displayed on the display part 210 of the display terminal 20. This is because when the difference time exceeds 50 msec, it is possible that a delay is occurring in the ultrasound image being displayed, and the puncture needle is accidentally inserted into the nerve depending on the puncture speed. When a transmission delay is occurring, the controller 200 causes the display part 210 to display delay information on the screen of the display part 210 in order to alert the user (Step S31).

FIG. 7B illustrates an example of the screen of the display part 210 of the display terminal 20 when the transmission delay exceeds the allowable range. Note that the basic display configuration of the screen is the same as that shown in FIG. 7A, and therefore the detailed description thereof will not be repeated.

When the difference time exceeds 50 msec, the controller 200 causes the display part 210 to display delay information in order to alert the user that a transmission delay is occurring. Specifically, when the difference time exceeds the allowable range, the bar B of the level meter 213 moves in an area on the right side of the center, for example. In this case, the bar B of the level meter 213 may be displayed in, for example, orange, red, or the like in order to call the user's attention.

The controller 200 may display, for example, text information 215 of “Delay is Occurring” on the right side of the level meter 213. The text information 215 may be displayed in different characters according to the degree of the transmission delay. For example, when the transmission delay is large, “Large Delay” may be displayed, whereas when the transmission delay is slightly larger than the normal, “Medium Delay” may be displayed. The controller 200 may cause a caution mark 216 to be displayed so as to be overlapped on the clock icon 212. Furthermore, the controller 200 may cause a frame 217 surrounding the periphery of the display area 211 to be displayed. The color of the frame 217 may be changed according to the degree of the transmission delay. For example, when the transmission delay is large, the color of the frame 217 may be yellow, red, or the like, or the frame 217 may be blinked.

The controller 200 determines whether the puncture process has finished (Step S32). For example, when an operation to end the puncture process is made, for example, a puncture end button is selected, the controller 200 may determine that the puncture process has finished. The controller 200 may determine that the puncture process has finished when a power button is turned off or when order information on the next patient is received.

When determining that the puncture process has finished, the controller 200 ends the series of processes. On the other hand, when determining that the puncture process has not finished, the controller 200 returns to Step S25. In this case, the controller 200 determines, for example, whether there is a transmission delay in the next frame to be transmitted from the display terminal 20.

### Sequence Diagram at Time of Pre-scanning and Puncture Process

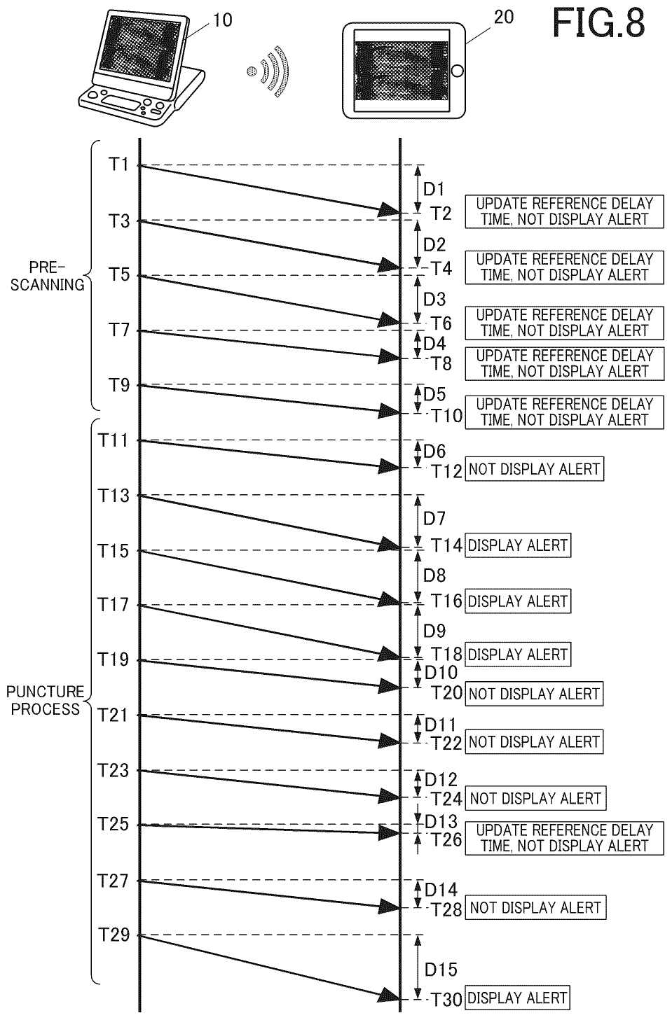

FIG. 8 is a sequence diagram showing the flow at the time of the pre-scanning and the puncture process in the diagnostic apparatus 10 and the display terminal 20. In the diagnostic apparatus 10 and the display terminal 20, the time on the diagnostic apparatus 10 side is synchronized with the time on the display terminal 20 side in advance.

First, pre-scanning is performed. At time T1, the diagnostic apparatus 10 generates ultrasound image data and a time stamp of the first frame. The diagnostic apparatus 10 transmits the generated ultrasound image data and time stamp to the display terminal 20. At time T2, the display terminal 20 receives the ultrasound image data and the time stamp of the first frame transmitted from the diagnostic apparatus 10. The display terminal 20 calculates delay time D1 of the first frame by subtracting the time T1 indicated by the time stamp from the time T2 that is the reception time. The display terminals 20 sets the calculated delay time D1 of the first frame as the reference delay time. The reference delay time is stored in the storage section 230, for example. The main purpose of the pre-scanning is to set the reference delay time, image parameters, and the like. Therefore, in the pre-scanning, even if a transmission delay is occurring, no delay information is displayed on the display part 210.

At time T3, the diagnostic apparatus 10 generates ultrasound image data and a time stamp of the second frame. The diagnostic apparatus 10 transmits the generated ultrasound image data and time stamp to the display terminal 20. At time T4, the display terminal 20 receives the ultrasound image data and the time stamp of the second frame transmitted from the diagnostic apparatus 10. The display terminal 20 calculates delay time D2 of the second frame by subtracting the time T3 indicated by the time stamp from the time T4 that is the reception time. The display terminal 20 updates the reference delay time by the calculated delay time D2 of the second frame. If the delay time D2 of the second frame is equal or substantially equal to the delay time D1 of the first frame, the delay time calculated for the previous frame may be used as the reference delay time without updating it.

In the present embodiment, regarding each of the third to fifth frames, a transmission delay of the same or substantially the same time as that of the first frame is occurring. Thus, the display terminal 20 calculates the delay time by the above-described method, and sequentially updates the reference delay time based on the calculated delay time.

When the pre-scanning finishes, the puncture process is performed. At time T11, the diagnostic apparatus 10 generates ultrasound image data and a time stamp of the sixth frame. The diagnostic apparatus 10 transmits the generated ultrasound image data and time stamp to the display terminal 20. At time T12, the display terminal 20 receives the ultrasound image data and the time stamp of the sixth frame transmitted from the diagnostic apparatus 10. The display terminals 20 calculate delay time D6 of the sixth frame by subtracting the time T11 indicated by the time stamp from the time T12 that is the reception time. The display terminal 20 compares the calculated delay time D6 of the sixth frame with the delay time D5 set as the reference delay time. In the present embodiment, the delay time D6 is equal to or substantially equal to the delay time D5. In this case, the display terminal 20 does not update the reference delay time. Since the transmission delay is within the allowable range, the display terminal 20 does not display the delay information on the display part 210.

At time T13, the diagnostic apparatus 10 generates ultrasound image data and a time stamp of the seventh frame. The diagnostic apparatus 10 transmits the generated ultrasound image data and time stamp to the display terminal 20. At time T14, the display terminal 20 receives the ultrasound image data and the time stamp of the seventh frame transmitted from the diagnostic apparatus 10. The display terminal 20 calculates delay time D7 of the seventh frame by subtracting the time T13 indicated by the time stamp from the time T14 that is the reception time. The display terminal 20 compares the calculated delay time D7 of the seventh frame with the delay time D5 set as the reference delay time. In the present embodiment, the delay time D7 is longer than the delay time D5. Therefore, the display terminal 20 does not update the reference delay time. Meanwhile, since the transmission delay exceeds the allowable range, the display terminal 20 displays the delay information indicating that a transmission delay is occurring on the display part 210.

In the present embodiment, regarding each of the eighth and ninth frames, a transmission delay of the same or substantially the same time as that of the seventh frame is occurring. Therefore, the display terminal 20 displays the delay information indicating that a transmission delay is occurring on the display part 210. Meanwhile, the delay time D8 of the eighth frame and the delay time D9 of the ninth frame are each equal to or substantially equal to the delay time D7 of the seventh frame. Therefore, the display terminal 20 does not update the reference delay time at the eighth frame and the ninth frame.

At time T19, the diagnostic apparatus 10 generates ultrasound image data and a time stamp of the tenth frame. The diagnostic apparatus 10 transmits the generated ultrasound image data and time stamp to the display terminal 20. At time T20, the display terminal 20 receives the ultrasound image data and the time stamp of the tenth frame transmitted from the diagnostic apparatus 10. The display terminals 20 calculate delay time D10 of the tenth frame by subtracting the time T19 indicated by the time stamp from the time T20 that is the reception time. The display terminals 20 compares the calculated delay time D10 of the tenth frame with the delay time D5 set as the reference delay time. In the present embodiment, the delay time D10 is equal to or substantially equal to the delay time D5. Therefore, the display terminal 20 does not update the reference delay time. Further, since a significant transmission delay is not occurring and the transmission delay is within the allowable range, the display terminal 20 hides the delay information that has been displayed on the display part 210.

In the present embodiment, regarding each of the eleventh and twelfth frames, a transmission delay of the same or substantially the same time as that of the tenth frame is occurring. Since a significant transmission delay is not occurring, the display terminal 20 does not display the delay information on the display part 210. Meanwhile, the display terminal 20 does not update the reference delay time at the eleventh frame and the twelfth frame.

At time T25, the diagnostic apparatus 10 generates ultrasound image data and a time stamp of the thirteenth frame. The diagnostic apparatus 10 transmits the generated ultrasound image data and time stamp to the display terminal 20. At time T26, the display terminal 20 receives the ultrasound image data and the time stamp of the thirteenth frame transmitted from the diagnostic apparatus 10. The display terminals 20 calculates delay time D13 of the thirteenth frame by subtracting the time T25 indicated by the time stamp from the time T26 that is the reception time. The display terminal 20 compares the calculated delay time D13 of the thirteenth frame with the delay time D5 set as the reference delay time. In the present embodiment. the delay time D13 is shorter than the delay time D5. Therefore, the display terminal 20 updates the reference delay time by the delay time D13 of the thirteenth frame. Meanwhile, since almost no transmission delay is occurring and the transmission delay is within the allowable range, the display terminal 20 continues not to display the delay information.

In the present embodiment, regarding the display image data of the fourteenth frame, a transmission delay is occurring. Further, delay time D14 of the fourteenth frame is equal to or substantially equal to the delay time D13 of the thirteenth frame. Therefore, the display terminal 20 does not update the reference delay time at the fourteenth frame. Further, since the transmission delay is within the allowable range, the display terminal 20 continues not to display the delay information.

At time T29, the diagnostic apparatus 10 generates ultrasound image data of the fifteenth frame and a time stamp. The diagnostic apparatus 10 transmits the generated ultrasound image data and time stamp to the display terminal 20. At time T30, the display terminal 20 receives the ultrasound image data of the fifteenth frame and the time stamp transmitted from the diagnostic apparatus 10. The display terminals 20 calculates delay time D15 of the display image data of the fifteenth frame by subtracting time T29 indicated by the time stamp from time T30 that is the reception time. The display terminal 20 compares the calculated delay time D15 of the fifteenth frame with the delay time D13 set as the reference delay time. In the present embodiment, the delay time D15 is longer than the delay time D13. Therefore, the display terminal 20 does not update the reference delay time. Meanwhile, since a transmission delay that exceeds the allowable range is occurring, the display terminal 20 displays the delay information on the display part 210.

There may be a case where a small number of frames has a transmission delay and the display time of the delay information is short. In this case, in order to reliably notify the user that a transmission delay is occurring, the delay information may be displayed on the display part 210 for a preset time.

Further, in the above-described embodiment, the time stamp is added to the ultrasound image data for each frame. For example, the time stamp may be added to the ultrasound image data for each unit of a plurality of frames or each preset time.

In addition, in the above-described embodiment, the delay information is displayed on the display part 210 in order to notify the user of the occurrence of the transmission delay, but this is not a limitation. The display terminal 20 may be provided with a speaker, and the user may be notified that a transmission delay is occurring by voice or a buzzer sound from the speaker. The sound or the like output from the speaker may be changed according to the degree of the transmission delay. Furthermore, the display terminal 20 may be provided with a vibration function so as to notify the user that a transmission delay is occurring by vibration. The vibration may be varied according to the degree of the transmission delay.

According to the present embodiment, the display terminal 20 displays the delay information indicating that the transmission delay of the ultrasound image data is occurring on the display part 210 of the display terminal 20. Thus, the user can recognize that a transmission delay of ultrasound image data exceeding an allowable range is occurring at the time of the puncture process. As a result, since the puncture can be performed in consideration of the transmission delay, the puncture can be accurately and quickly performed, and a medical error caused by the transmission delay can be prevented.

Furthermore, according to the present embodiment, the display terminal 20 compares the delay times, which are relative values of the respective frames, with each other, and determines whether or not a transmission delay is occurring. Therefore, it is possible to accurately determine whether a transmission delay is occurring or whether the transmission delay is within an allowable range. Thus, puncturing can be performed accurately and quickly, and medical errors due to transmission delay can be prevented.

Further, according to the present embodiment, when the delay time during the puncture process becomes larger than the reference delay time set during the pre-scanning, the delay information is displayed to the user. In other words, when the situation due to the transmission delay deteriorates during the puncture process, the delay information is displayed to the user. Thus, a user can accurately recognize, from the display of the delay information, that a transmission delay exceeding an allowable range is occurring.

Although one or more preferred embodiments of the present disclosure have been described in detail with reference to the accompanying drawings, the technical scope of the present disclosure is not limited to such examples, Furthermore, those to which various modifications and improvements have been applied naturally belong to the technical scope of the present disclosure within the category of the technical ideas described in the scope of claims.

For example, the difference between the transmission time of the image data on the diagnostic apparatus 10 side and the reception time of the image data on the display terminal 20 side may be set as the difference time. In this case, the display terminal 20 determines whether or not to display delay information indicating that a transmission delay is occurring, based on the calculated difference time. In order to eliminate a deviation between the time on the diagnostic apparatus 10 side and the time on the display terminal 20 side, the time on the diagnostic apparatus 10 side and the time on the display terminal 20 side are synchronized.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2023-101399, filed on Jun. 21, 2023, including description, claims, drawings and abstract is incorporated herein by reference.

What is claimed is:

1. An ultrasound diagnostic system comprising:
an ultrasound diagnostic apparatus that generates image data; and
an ultrasound image display terminal that is a separate unit from the ultrasound diagnostic apparatus and displays an ultrasound image based on the image data;
wherein the ultrasound diagnostic apparatus and the ultrasound image display terminal are connected with one another via a wireless network, and
wherein the ultrasound diagnostic system further comprises:
a transmitter that transmits the image data from the ultrasound diagnostic apparatus to the ultrasound image display terminal; and
a hardware processor that outputs delay information indicating that a transmission delay of the image data is occurring.

2. The ultrasound diagnostic system according to claim 1, wherein the hardware processor
determines whether the transmission delay is occurring, and
outputs the delay information based on a result of the determination.

3. The ultrasound diagnostic system according to claim 2, wherein the hardware processor outputs the delay information in response to a predetermined condition being satisfied.

4. The ultrasound diagnostic system according to claim 3, wherein the predetermined condition is a puncture mode.

5. The ultrasound diagnostic system according to claim 1,
wherein the delay information is delay information indicating that the transmission delay is occurring at at least one of the ultrasound diagnostic apparatus and the ultrasound image display terminal, and
wherein the hardware processor
acquires reference delay time information acquired at a first time and second delay time information acquired at a second time different from the first time, and
calculates, based on the reference delay time information and the second delay time information, a difference time indicating whether the transmission delay is occurring.

6. The ultrasound diagnostic system according to claim 5, wherein the hardware processor outputs the delay information in response to the difference time being equal to or greater than a predetermined threshold value.

7. A non-transitory computer-readable storage medium storing a program that causes an ultrasound image display terminal that is a separate unit from an ultrasound diagnostic apparatus and is connected with the ultrasound diagnostic apparatus via a wireless network, the ultrasound diagnostic apparatus generating image data, to perform:
receiving the image data transmitted from the ultrasound diagnostic apparatus;
displaying an ultrasound image based on the image data; and
outputting delay information indicating that a transmission delay of the image data is occurring.

8. A non-transitory computer-readable storage medium storing a program that causes an ultrasound diagnostic apparatus that is a separate unit from an ultrasound image display terminal, is connected with the ultrasound image display terminal via a wireless network, and generates image data, the ultrasound image display terminal displaying an ultrasound image, to perform:
transmitting the image data to the ultrasound image display terminal; and
outputting, to the ultrasound image display terminal, time information for calculating delay information indicating that a transmission delay of the image data is occurring.

9. An ultrasound image display terminal that is a separate unit from an ultrasound diagnostic apparatus and is connected with the ultrasound diagnostic apparatus via a wireless network, the ultrasound diagnostic apparatus generating image data, comprising:
a receiver that receives the image data transmitted from the ultrasound diagnostic apparatus;
a display that displays an ultrasound image based on the image data; and
a hardware processor that outputs delay information indicating that a transmission delay of the image data is occurring.

10. An ultrasound diagnostic apparatus that is a separate unit from an ultrasound image display terminal, is connected with the ultrasound image display terminal via a wireless network, and generates image data, the ultrasound image display terminal displaying an ultrasound image, comprising:
a transmitter that transmits the image data to the ultrasound image display terminal; and
a hardware processor that outputs, to the ultrasound image display terminal, time information for calculating delay information indicating that a transmission delay of the image data is occurring.

11. The ultrasound diagnostic apparatus according to claim 10, comprising a storage that stores a program that causes the ultrasound image display terminal to perform:
receiving the image data transmitted from the ultrasound diagnostic apparatus;
displaying an ultrasound image based on the image data; and
outputting the delay information indicating that the transmission delay of the image data is occurring,
wherein the hardware processor outputs the program to the ultrasound image display terminal.
